(19) United States
(12) Patent Application Publication
(10) Pub. No.: US 2012/0232348 A1
(43) Pub. Date: Sep. 13, 2012

(54) Disposable Medical Fiber Optic Light Guide Tube

(76) Inventor: Millie Martinez, Placentia, CA (US)

(21) Appl. No.: 13/411,748

(22) Filed: Mar. 5, 2012

Related U.S. Application Data
(60) Provisional application No. 61/449,992, filed on Mar. 7, 2011.

(51) Int. Cl. A61B 1/07

(52) U.S. Cl. 600/178

(57) ABSTRACT

A disposable attachment to a medical inspection or speculum device is disclosed that improves control of a fiber optic light source utilized in conjunction with the medical device to provide illumination within an internal body cavity. The attachment comprises a hollow guide tube and support having an attachment mechanism, wherein the attachment is adapted to affix to a medical device while the guide tube allows the fiber optic light to be fed therethrough and into the body cavity of interest. The guide tube provides physicians with a readily attachable and disposable alternative to existing methods of handling light sources, which often require diligent guidance and sterilization after each patient. In use, the device allows physicians to accurately inspect and diagnose conditions within a cavity by providing a stable light source to an existing instrument, whereafter the guide tube may be removed and discarded, and further the light may remain sterile.

### CROSS REFERENCE TO RELATED APPLICATION

This application claims the benefit of U.S. Provisional Application No. 61/449,992 filed on Mar. 7, 2011, entitled “Fiberoptic Lited Vaginal Speculum.”

### BACKGROUND OF THE INVENTION

1. Field of the Invention

The present invention relates to fiber optic light sources and medical instruments. More specifically, the present invention pertains to a medical fiber optic light guide tube that provides improved control of a fiber optic light source for medical examinations within body cavities. The guide tube is affixable to a medical instrument and provides a guide tube in which a fiber optic light is placed, controlling the light's position with regard to the instrument and facilitating improved light source control during examinations. The light source is kept sterile during the examination, whereafter the guide tube is removable from the medical device and can be discarded and replaced. The primary medical instrument contemplated by the present invention is a vaginal speculum and for improving visualization related to vaginal examinations therewith.

A speculum is a medical device that allows a physician to visually inspect an internal body cavity by separating an opening to allow visual access thereinto. The speculum comprises a handheld device having a plurality of ‘blades’ to separate an opening to a body cavity and allow direct vision between the blades and into the cavity of interest. Examinations of this type can be facilitated in conjunction with a light source, preferably a fiber optic light that can be placed into the body cavity for direct illumination. While such light sources provide a means to illuminate the cavity, they generally require the examiner to hold both the speculum and the light source simultaneously, while at the same time conducting the examination. This can be a cumbersome activity given the needs of the examiner to visually expect and then conduct any medical procedures necessary during the examination. The present invention is provided to improve handling and control of such a light source in conjunction with any handheld medical device, wherein the fiber optic end of the light may be fed through the guide tube, which is attached to the medical device to free the hands of the physician or medical professional during examinations or medical procedures.

Another issue during medical examinations is the need to keep all instruments and tools that contact the patient sterile. This prevents the spread of disease and the possibility of infection from contaminated objects coming into contact with open wounds or internal cavities. Generally all instruments are sterilized prior to a procedure, and once utilized are thereafter re-sterilized or discarded. Larger, complex or more expensive tools are reused and thus re-sterilized after each use. This can be time consuming, laborious and expensive depending on the instrument and the number of redundant instruments available for repeat procedures. The present invention provides a means to prevent a fiber optic light source from contacting a given patient during an examination or procedure by shrouding its optical working end within a hollow guide tube. The light is fed through the guide tube and removed after use, while the guide tube itself can be separated from the medical instrument and discarded rather than be sterilized and reused. The relatively low cost of each guide tube makes the present invention an ideal tool in place of re-sterilization procedures for medical fiber optic lights, which would otherwise be required.

The disclosed invention attempts to remedy these known drawbacks to traditional devices and methods of illuminating internal cavities and handling light sources in conjunction with other necessary medical devices. The present invention is a disposable, attachable medical accessory that clips or otherwise attaches to the medical device and allows the light source to be integrally held against the device while keeping the light source sanitary between patients. The device is useful for a number of medical devices, and is particularly suited for speculums during internal cavity examinations.

2. Description of the Prior Art

Lighted medical devices and fiber optic light sources related to the present invention have been disclosed and patented in the prior art. These devices have familiar design features for the purposes of providing improved illumination within an interior body cavity during a medical examination or procedure. Most of these are related to combination medical devices that incorporate a light source into their design or further facilitate the use of a fiber optic light source by a medical professional. These devices, however, fail to disclose the novel elements of the present invention. In particular, the disposable nature thereof and its ability to affix to most any medical tool and provide a means to stabilize and maintain sterilization of a fiber optic light working end is not disclosed in the prior art.

Specifically, U.S. Pat. No. 5,394,863 to Sanford describes a vaginal fornix illuminator comprising a light transmisssive plastic pipe body having an elongated stem and hollow cup. The cup is adapted to engage over the cervix as light is transmitted through the cup and into the vaginal fornix to improve vision during medical procedures. The free end of the pipe body is adapted to accept a light source that transmit light into the patient's body cavity to improve a medical professional's vision within the patient. While providing a novel device that is particularly suited for engaging a female cervix, the Sanford device fails to disclose a device that works in conjunction with a speculum or similar medical device to shroud and guide a fiber optic light source as provided by the present invention.

U.S. Pat. No. 4,597,383 to VanDerBel is another such device that discloses a combination vaginal speculum and fiber optic illumination means. The device comprises speculum connected to a working end of a fiber optic light by means of an L-shaped mounting bracket, wherein the bracket provides connection between the two independent devices. The light is held at the base of the speculum blades, providing improved illumination within a body cavity wherein the speculum is providing a means to spread the body cavity opening. While the VanDerBel device provides a light source attachment means for a vaginal speculum, it fails to contemplate the structure of intent of the present invention. The present invention provides both a guide and shroud, wherein the light source may enter the patient's body cavity and be preventing from being contaminated via contact therewith. The guide tube allows the working end of the fiber optic light source to remain sterile during the procedure, whereafter the guide tube is detached and discarded.

Similar to the VanDerBel device is U.S. Pat. No. 6,379,296 to Baggett, which discloses a light source that is particularly suited for medical examination devices and in particular a vaginal speculum. The light itself comprises an elongated and flexible arm that is adapted to be fed through tubes commonly utilized in medical, dental and veterinary procedures. The light is attachable to the speculum using an attachment means that affixes to the speculum, which generally comprises a plurality of brackets to support the light power supply and a plastic sheath along the speculum blade that covers the light arm. The Baggett device employs a speculum having a plurality of support brackets and an attached covering sheath, while also providing a new and improved light source that is particularly adapted for use with the speculum. The present invention contemplates the use any fiber optic light source, while its structure provides a means to sheath and guide the working end of the light source while connected to any medical device that facilitates internal examinations or procedures.

U.S. Pat. No. 4,905,670 to Adair discloses another combination device that comprises a videoscope attached to the proximal end of a pair of speculum blades. The attachment comprises a track mounted longitudinally along one of the speculum blades, providing support for the both the videoscope and a light carrier for illuminating a patient's cervix while in operation. The position of the videoscope along the track is controllable via a worm gear that allows longitudinal motion thereof. This device is adapted for treating lesions on the patient's cervix, wherein a means for supporting instruments therefor along a speculum is disclosed. The entire assembly must be sterilized between patients, as the videoscope is not shrouded from contact with the patient or adjacent fluid. The present invention diverges in spirit and design from the Adair disclosure, which is particularly suited for its intended use of treating lesions and other abnormalities on a patient's cervix.

U.S. Pat. No. 4,337,763 to Petrassevich discloses a medical implement having improved functional configuration that provides a means for illuminating the interior of a body cavity formed by an incision or wound. The device comprises a substantially straight blade portion having a shank portion in perpendicular alignment thereto and providing a handle. An illumination means is provided toward the tip of the blade, comprising a bulb and socket, wherein the bulb is shrouded by a flared upper wall. Electrical components necessary to operate the bulb are provided within the shank handle portion, including battery power and an electrical switch means. The Petrassevich provides a novel retractor implement with imbedded illumination means, however this device fails to contemplate the use of a fiber optic lighting element and guide means therefor.

The present invention provides an attachment to a medical device that allows the working end of a fiber optic light to be held in a static position while freeing the hand of the physician or medical professional to conduct an examination of an internal body cavity. The device comprises a guide tube that attaches to a planar surface of a medical device, wherein the guide tube controls the light and also shrouds its working end to maintain its sterility during the procedure. The devices in the prior art relate to combination light and speculum devices with varying elements related to the present invention, yet they fail to disclose a detachable and disposable guide tube having the novel aspects disclosed herein. The present invention is substantially divergent in design elements from the prior art, consequently it is clear that there is a need in the art for an improvement to existing medical tool light attachments. In this regard the instant invention substantially fulfills these needs.

### SUMMARY OF THE INVENTION

In view of the foregoing disadvantages inherent in the known types of medical device attachments now present in the prior art, the present invention provides a new attachment wherein the same can be utilized for providing convenience for the user when supporting and shrouding the working end of a fiber optic light source in conjunction with a medical instrument, and in particular a speculum device.

It is therefore an object of the present invention to provide a new and improved medical device attachment that has all of the advantages of the prior art and none of the disadvantages.

It is another object of the present invention to provide a disposable and universally attachable guide means for a fiber optic light source that operates in conjunction with a medical internal body cavity inspection and examination device.

Another object of the present invention is to provide a medical guide tube attachment that is adapted to support the working end of a fiber optic guide tube in a static position in connection with a medical device.

Yet another object of the present invention is to provide a medical guide tube attachment that is adapted to maintain the sterility of a fiber optic light source working end when in connection with a body cavity inspection or examination device.

A final object of the present invention to provide a medical guide tube attachment that employs an elongated guide tube construction attaching to a base having an attachment mechanism adapted to connect to any planar surface provided on a medical device.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a side view of a particular embodiment of the present guide tube device.

FIG. 2 shows a perspective view of the first embodiment of the present guide tube device.

FIG. 3 shows a side view of a second embodiment of the present guide tube device.

FIG. 4 shows a side view of yet another embodiment of the present guide tube device.

FIG. 5 shows a side view of the present invention in connection with a speculum device.

FIG. 6 shows an underside perspective view of the present invention in a working position and in connection with a speculum device.

### DETAILED DESCRIPTION OF THE INVENTION

Reference is made herein to the attached drawings. Like reference numerals are used throughout the drawings to depict like or similar elements of the medical device attachment. For the purposes of presenting a brief and clear description of the present invention, the preferred embodiment will be discussed as used for providing support for a fiber optic light source in connection with a medical instrument and providing sterile coverage thereover. The figures are intended for representative purposes only and should not be considered to be limiting in any respect.

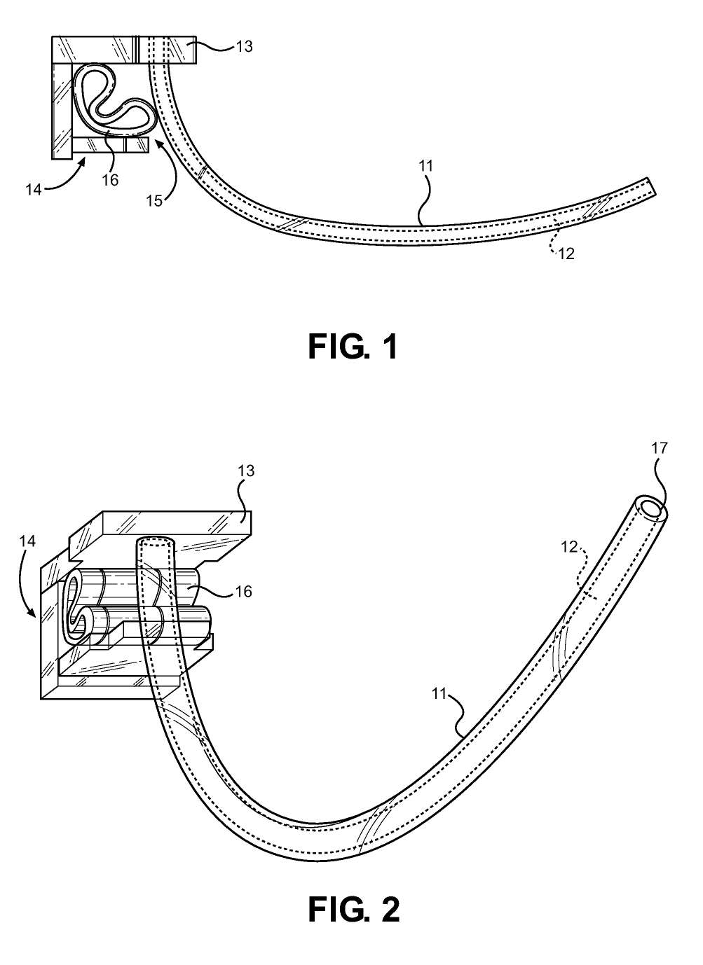

Referring now to FIG. 1, there is shown a side view of a first embodiment of the fiber optic light source guide tube of the present invention. The guide tube 11 is an elongated, arcuate and hollow member having an interior cavity 12 adapted to securely contain the working end of a fiber optic light source. The fiber optic light is fed through the tube interior 12 up to its distal end. The distal end may provide either an opening to facilitate improve lighting, or alternatively be closed off to improve sterilization of the light by preventing contact with a patient during a procedure. The proximal end of the guide tube 11 mates to a platform 13, wherein the tube interior 12 extends through the platform 13 to provide an aperture through which to begin feeding of the fiber optic light. The platform 13 is a planar surface having a thickness, wherein a medical device attachment means 14 is further positioned thereon.

The attachment means 14 of the present invention may take several forms for the function of attaching the guide tube 11 and platform 13 to a free attachment surface on a given medical device. In a particular embodiment, the attachment means is an enclosed compression element 16 wherein the attachment surface of the medical device is slid into a groove 15 between the guide tube 11 and the compression element 16 such that the element 16 is compressed and forms a secure joint with the medical device free surface. The free surface is thus compressed on both sides thereof, first by the proximal end of the guide tube 11 and secondly by the compression element 16 bearing thereagainst. The compression element 16 is any elastic element with stiffness, adapted to provide a counteracting load upon compression from an initial position. In this embodiment, the compression element 16 is a tubular element having a central kink, wherein the cross section of the element is compressible when the free surface of the medical device is pressed into the groove 15 provide behind the guide tube 11. In this configuration, the guide tube 11 rests over the medical device free surface, which may be suitable depending on the application, desired position of the fiber optic light source and the structure of medical device.

Referring now to FIG. 2, there is shown an underside perspective view of the first embodiment of the present fiber optic guide tube device of the present invention. This embodiment employs an attachment means 14 that comprises the tubular compression element 16 housed within the body structure of the device, wherein the guide tube 11 itself is attached to a platform base 13 for which a fiber optic light is fed through and into the hollow interior 12 of the guide tube. As visualized, the guide tube is a arcuate member and is comprised of a flexible material that allows repositioning with regard to the given medical instrument thereattached, while also providing inherent stiffness to provide a stable support for the working end of the light. This ensures that the light is positioned as desired along the medical instrument prior to and during the examination or medical procedure. The light can be fed through the tube 12 along its length and up to its distal end 17. This distal end 17 may provide an opening such to allow a directed stream of light, or preferably remain closed off to retain sterilization of the fiber optic light working end during a procedure.

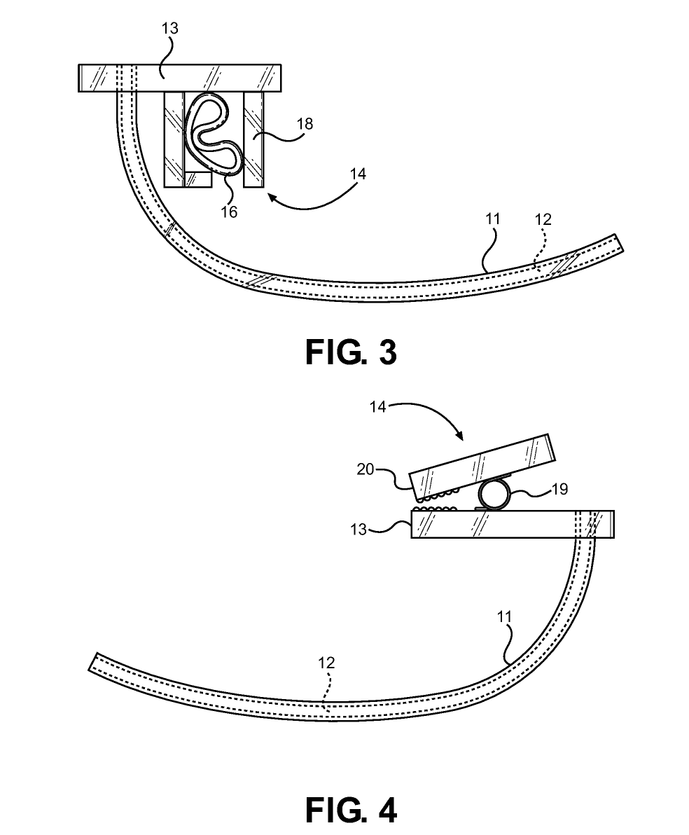

Referring now to FIG. 3, there is shown a side view of an embodiment of the fiber optic guide tube device, wherein an alternative attachment means 14 and guide tube 11 configuration is provided. In this embodiment, the free surface of the medical instrument for which the present device is adapted to attach is positioned above the guide tube 11 and tube slides thereunder. The free surface is fed between a compression element 16 and an adjacent wall 18 that projects from the platform base 13 of the guide tube assembly. The compression element 16 compresses the surface against the adjacent wall 18 to secure the entire assembly and prevent movement, while the guide tube 11 extends from behind the attachment means 14 rather than forward thereof. Similar to the first embodiment, the guide tube interior 12 extends through the base 13 to allow a fiber optic light source to be fed therethrough and along the length of the guide tube 11 up to its distal end, which may be closed or be opened depending on requirements.

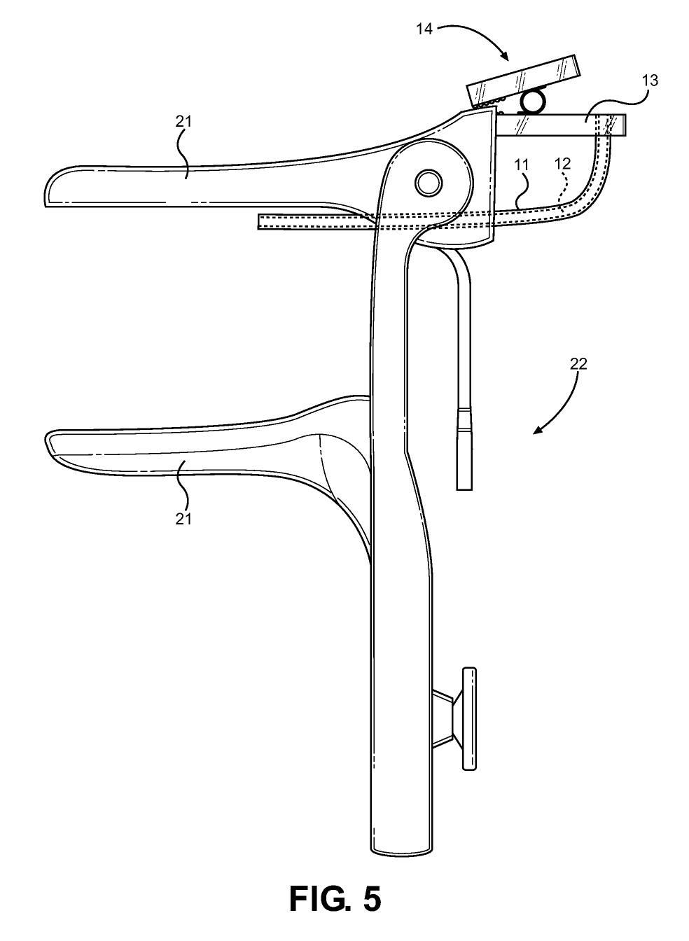

Referring now to FIG. 4, there is shown a side view of the present invention, including another contemplated embodiment of the present attachment means 14 of the guide tube device. In this configuration, a spring clip is provided that attaches to the platform base 14 of the assembly and allows a free surface of a medical instrument to be secured between a first jaw member 20 and the upper or lower surface of the platform 13. A helical spring 19 is provided between the jaw member 20 and the platform, wherein the spring 19 provides a resistance against the jaw 20 separating from the platform 13 at their junction point, which also functioning as a fulcrum for which to rotate the jaw 20 away from the platform 20 and allow for an opening to engage the medical device of interest and of which the device is being attached. Once attached, the elongated guide tube 11 is positioned to allow control and sterile deployment of the working end of a fiber optic light source.

Referring now to FIG. 5, there is shown a side view of the present invention attached to a speculum medical device 22, wherein the attachment means 14 attaches to a free surface along the proximal end of one of the speculum's blades 21. This allows access to the device guide tube 11 for insertion of a fiber optic light source and also allows a medical professional to operate the speculum 22 without holding the light source during an examination. The guide tube 11 positions an inserted fiber optic light between the speculum blades and into a body cavity of interest for improved illumination of the area of interest. The platform 13 of the present invention provides a stable ledge for which to allow the working end of a fiber optic light to be fed into the hollow interior 12 of the guide tube. The attachment means 14 is one in which a free surface is compressed within two members, allowing the device to remain firmly in place on the medical device 22 while conducting a medical operation or examination.

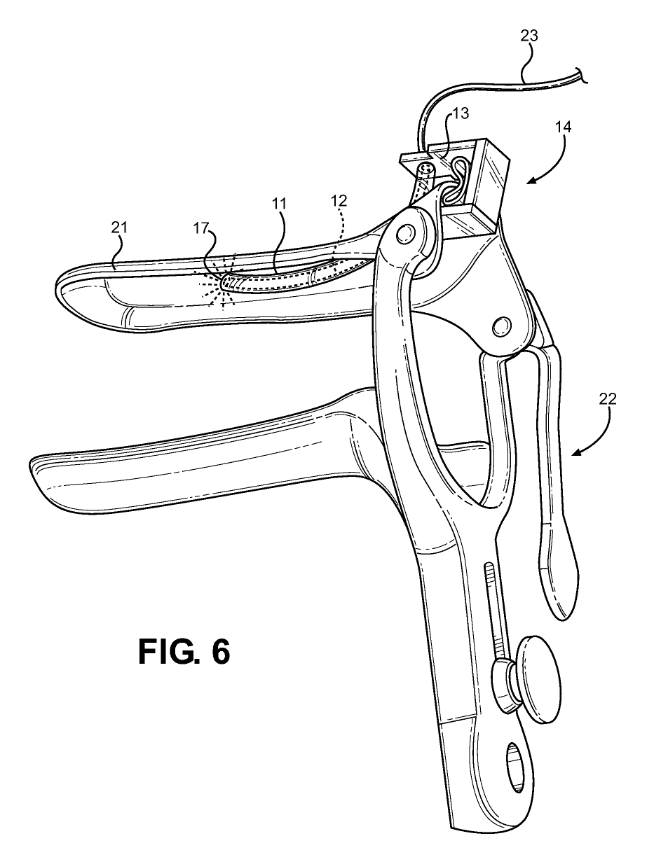

Referring now to FIG. 6, there is shown a rear perspective view of the present invention in use and attached to an upper blade 21 of a speculum device 22. The first embodiment of the attachment means 14 is deployed, wherein the free surface of the speculum blade 21 along its proximal end is sandwiched between the compressible member and the guide tube 11, wherein the tube feeds through an aperture in the speculum blade 21 and rests in a position between the two opposing blades 21. This positioning allows a fiber optic light source 23 to be fed through the guide tube 11 and illuminate an area between the separated blades 21 within a patient interior cavity. The medical professional is free to operate the speculum without holding the working end of the fiber optic light 23, as it is securely positioned against the speculum during the procedure.

It is desired to disclose a readily attachable and readily disposable light guide tube that is capable of attachment onto the free surface of a medical device, wherein the tube provides a stable and sterile covering for the working end of a fiber optic light source. The device comprises an elongated and sufficiently stiff yet still elastic guide tube exhibiting an arcuate and easily malleable configuration, which is attached at its proximal end to a base having a removable attachment means. The attachment means may take several forms, and it is contemplated that it include any suitable clip or compression fitting such that the it is affixable to the free surface of a given medical device. The distal end of the guide tube is preferably closed to improve sterility; however this end may also be opened to provide a light-directing tip. In use, the working end of a fiber optic light is fed through the hollow guide tube and along its length, wherein the tube is affixed to a medical device such that the light remains securely attached and sterile during a procedure. After the device is utilized and the light is removed, the guide tube is detached and discarded thereafter. The light remains sterile while the guide tube is readily disposable. Overall, the device is an inexpensive alternative to re-sterilization procedures for the light source, wherein the light must be placed in a condition of un-use and sterilized before being utilized on the next patient. The present device is further contemplated for use with any medical device having a free ledge surface for which to attach and for which is required improved illumination.

In light of the forgoing description and the disclosed prior art, it is herein submitted that the instant invention is sufficiently differentiated from the prior art and has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A readily disposable fiber optic light source guide tube, comprising:
an elongated and tubular guide tube having a hollow interior, a distal and proximal end;
said guide tube proximal end connected to a base platform having an aperture through which to communicate a fiber optic light source through said platform and into said guide tube hollow interior;
an attachment means connected to said platform base adapted to affix to a free surface edge.

2. The device of claim 1, wherein said guide tube distal end is closed to prevent contamination of said fiber optic light source.

3. The device of claim 1, wherein said guide tube distal end provides an open aperture to allow a directed illumination from a fiber optic light source.

4. The device of claim 1, wherein said elongated and tubular guide tube is of sufficient stiffness to maintain a fiber optic light source work end in a static position, while allowing forceful repositioning thereof by a user.

5. The device of claim 1, wherein said elongated and tubular guide tube is provided in a malleable arcuate shape.

6. The device of claim 1, wherein said guide tube, base platform and attachment means are readily disposable after a single use.

7. The device of claim 1, wherein said guide tube provides a sterile environment within its hollow interior to prevent contamination of said fiber optic light source.

8. The device of claim 1, wherein said attachment means further comprises a compression element adapted to compress said free surface between said element and said guide tube proximal end to form a secure joint.

9. The device of claim 1, wherein said attachment means further comprises:
a compression element and an adjacent surface;
said adjacent surface projecting from said base platform;
said compression element adapted to compress said free surface between said element and said adjacent surface to form a secure joint.

10. The device of claim 1, wherein said attachment means further comprises a clip.

11. The device of claim 1, wherein said attachment means further comprises a spring clip having a first jaw member and a connecting spring element to secure said free surface between said jaw member and said base platform.
